(19) United States
(12) Patent Application Publication
(10) Pub. No.: US 2008/0039508 A1
(43) Pub. Date: Feb. 14, 2008

(54) Treatment Of Tumours

(75) Inventors: Russell Taylor, Berkshire (GB); Paul Crees, Berkshire (GB)

Correspondence Address: MYERS BIGEL SIBLEY & SAJOVEC, PO BOX 37428, RALEIGH, NC, 27627, US

(73) Assignee: AQ+PLC, GLASGOW (GB)

(21) Appl. No.: 11/632,326

(22) PCT Filed: Jul. 15, 2005
(86) PCT No.: PCT/GB05/02759

§ 371 Date: Jul. 6, 2007

(30) Foreign Application Priority Data
Jul. 15, 2004 (GB) 0415768.1
Apr. 26, 2005 (GB) 0508411.6

(51) Int. Cl. A61K 31/47; A61P 31/04; A61P 35/00

(52) U.S. Cl. 514/357

(57) ABSTRACT

The present invention provides the use of a metal ion chelating agent for the manufacture of a medicament for the treatment or prophylaxis of a bacteria-mediated cancerous tumour. The metal ion chelating agent provides a metal ion chelating capacity for at least one metal ion on which the tumorigenic bacteria is dependent for viability. The invention also provides methods for the treatment or prophylaxis of tumours.

This invention relates to manufacture and applications of metal ion chelating compositions for the treatment of bacteria mediated cancerous lesions and tumours in human and non-human animals.

The debate still rages on about the source or the origin of cancerous conditions and after many years, in spite of substantive indications, it is still denied vigorously that bacteria may be the cause. Substantive work on bacteria causing cancer goes back to the 18^{th} century where it was even recognised then that a form of bacteria was probably the cause. With developments in recent years on other medical conditions it has been recognised that bacteria can change their shape (pleomorphism) and that with such changes, the bacteria can produce different medical conditions.

It has been observed that bacteria change from one form (rod shape) to another form (cocolith shape) and vice versa, and that under these circumstances the bacteria accumulates into lesions which then appear to become cancerous. Sometimes these lesions form what is called sarcoid lesions and in a lot of cases they can become quite extensive. It has been said that these lesions are eruptions from the autoimmune system reacting to localised circumstances. It does appear that the localised circumstances centre around pleomorphic bacteria.

With pleomorphism, when the bacteria changes shape it appears that there is no or only minimal cell wall structure, and when this happens the bacteria appears to coat itself with a biofilm—normally of calcium, to make itself impervious to antibiotics and other chemical compounds.

It also appears to be the case that when the bacteria is threatened by the immune system or by antibiotics they may lose their cell-wall and assume a different growth form that renders them less susceptible to attack by the immune system. This lack of cellular response is another reason why the medical profession has found difficulty in accepting that bacteria are the source of cancer.

It is now known that the bacteria Streptococcus pyogene, which was first mentioned in 1908 as being the reason for a large number of baby deaths on birth, and after 1945 was responsible for Scarlet Fever, is responsible for Rheumatic Fever and Necrotising fasciitis conditions. This bacteria has been shown to be pleomorphic and thus is able to change its properties and structure, and with this capability to give rise to numerous medical conditions.

It has also been shown that nano-bacteria, which are present in everyone and, it has also been recognised, are present in many vaccines as an impurity, are also pleomorphic and have a biofilm coating. This is one of the reasons why antibiotics and the body's immune system, cannot deal with them. With the calcium biofilm coating, the body assumes that the nano-bacterium is simply a piece of calcium moving about within the system. Also under certain conditions these nano-bacteria seem to accumulate and develop into lesions.

With this special property of pleomorphism attributed to bacteria as a potential precursor source of cancerous conditions, these bacteria have become a target for cancer tumour treatment. It appears that the bacteria may accumulate in more or less any part of the body and upon such accumulation, may develop into a tumour. Furthermore, the growth of the tumour also seems to be dependent on a ferric ion dependent bacteria, which is associated with pleomorphic bacteria.

Pleomorphic properties can be found in many different species of bacteria. Thus, for example, Staphylococcus epidermis, which is normally a non-pathogenic bacteria found on the skin or mucous membranes, has been located in breast tissue in rod and coccolithic forms and found to be creating a cancerous lesion. It has been shown that Mycobacterium sp. can also change form and produce various other diseases.

It is an object of the present invention to avoid or minimize one or more of the above problems.

There has previously been proposed in our earlier Patent publication WO 03/032944, various topical chelating compositions suitable for use in combatting antibiotic-resistant infections and contamination of the skin and open wounds. There has been no previous suggestion, however, of the possible utility of such compositions for other conditions.

We have now found that compositions based on the use of chelating compounds as disclosed in WO 03/032944 (the contents of which are hereby incorporated herein by reference thereto), have the properties of treating cancerous tumours, reducing them in size and reducing the amount of cancerous cells present over a period of time.

Thus in a first aspect the present invention provides the use of a metal ion chelating agent for the manufacture of a medicament for the treatment or prophylaxis of a bacteria-mediated cancerous tumour, wherein said metal ion chelating agent provides a metal ion chelating capacity for at least one metal ion on which said tumorigenic bacteria is dependent for viability.

The present invention also provides the use of a metal ion chelating agent for the manufacture of a medicament for the treatment or prophylaxis of a tumour selected from Karposi and Sarcoid tumours.

In a further aspect the present invention provides a method for the treatment or prophylaxis of a bacteria-mediated cancerous tumour, comprising the administration, to a human or animal in need of such treatment, of an effective dose of a metal ion chelating agent, wherein said metal ion chelating agent provides a metal ion chelating capacity for at least one metal ion on which said tumorigenic bacteria is dependent for viability.

Having regard to the presence of the calcium coating on a wide range of tumorigenic bacteria, we have found that in such cases it is also essential that there is used a chelating agent that will absorb calcium present in a calcium based coating on the tumorigenic bacterium membranes in order to make the bacterial cell available to be attacked by a chelating agent that has a chelating capacity for one or more metal ions necessary for the viability of the tumorigenic cell—such as Mg²⁺, Fe²⁺, Fe³⁺, Cu²⁺, Zn²⁺, Mn²⁺, Ni²⁺, and Se²⁺, which may conveniently be referred to as “nutrient” metal ions. Desirably also there is used a chelating agent that has a chelating capacity for ferric ions in order to attack the ferric ion dependent bacterial cells which support tumour growth. Suitable, calcium ion-chelating, agents are well known in the art, and include inter alia EDTA.

Thus in another aspect the present invention provides the use of at least one metal ion chelating agent for the manufacture of a medicament for the treatment or prophylaxis of a bacteria-mediated cancerous tumour, wherein said metal ion chelating agent provides a metal ion chelating capacity for calcium ions and for at least one metal ion on which said tumorigenic bacteria is dependent for viability.

In a further aspect the present invention provides a method for the treatment or prophylaxis of a bacteria-mediated cancerous tumour, comprising the administration, to a human or animal in need of such treatment, of an effective dose of at least one metal ion chelating agent, wherein said at least one metal ion chelating agent provides a metal ion chelating capacity for calcium ions and for at least one metal ion on which said tumorigenic bacteria is dependent for viability.

It is in principle possible to use a single chelating agent which provides two or more of the above essential or desirable requirements. In practice though we have found that different chelating agents have different chelating capability or strength for different metal ions, and it is generally necessary to use two or more different chelating agents wherein the chelating capability for the various different metal ion targets is maximized to a greater or lesser extent.

Having regard to the above, 8-hydroxyquinoline has been found to have a particularly broad spectrum of activity, chelating, with a high degree of effectiveness, most metal ions (including ferric), apart from sodium, potassium and calcium. It is accordingly necessary to use this chelating agent in combination with a secondary, calcium ion-chelating, agent. Suitable, calcium ion-chelating, agents are well known in the art, and include inter alia EDTA.

By providing a metal ion chelating agent that removes a variety of metal ions, which may conveniently be referred to as the target metal ions, the effectiveness of the compositions of the invention may be increased. Thus, in relation to attacking the basic “nutrient” metal ion requirement of the bacterial cell, it is preferable to use a metal ion chelating agent which can form a chelate with a plurality of metal ions selected from Mg²⁺, Fe²⁺, Fe³⁺, Cu²⁺, Zn²⁺, Mn²⁺, Ni²⁺, and Se²⁺.

It appears that at least part of the effect of the primary—nutrient metal ion, chelating agent is through a coating of the tumorigenic bacteria, by the primary chelating agent, thereby effectively smothering the bacteria. It also appears that the primary chelating agent has a similar smothering effect on the ferric ion dependent bacteria that appears to control tumour growth.

Either or both of the primary and secondary chelating, agents will also chelate free ions in the inter-cellular medium, making them unavailable to support bacterial growth and development. The resulting damage to the ferric ion mediated bacteria will then induce the tumour to start shrinking. With constant application of the chelating compound compositions of the invention over a period of time, this will continually deny the ferric ion mediated and pleomorphic tumorigenic bacteria, any food, until they are completely eliminated.

Another significant benefit of the chelating compound compositions of the invention, is that they coat nerve receptors in the tumour area and thereby reduce the pain experienced by the patient. This action also appears further to enhance the effectiveness of the treatment of the tumours or cancerous conditions.

Preferred chelating agents can chelate various different metal ions and thereby attack the tumorigenic bacteria by multiple, direct and indirect, routes. More particularly, it is preferable for the chelating agent(s) used to form a chelate with a plurality of metal ions selected from Mg²⁺, Fe²⁺, Cu²⁺, Zn²⁺, Mn²⁺, Ni²⁺, and Se²⁺. 8-hydroxyquinoline has been found to have a particularly broad spectrum of activity, chelating most metals apart from sodium, potassium and calcium.

Preferably the primary “nutrient” metal ion chelating agent is a heteropolar compound comprising at least one unsaturated heterocyclic six-membered ring in which at least one heteroatom moiety acts as a hydrogen acceptor and in which said compound also comprises at least one hydrogen donor moiety, conveniently a hydroxyl group, said heteropolar compound having no substituent which by itself or together with another substituent or substituents creates such steric hindrance and/or renders the molecule so basic or acidic or so alters the steric geometry of the molecule as to prevent interaction of the hydrogen donor and acceptor moieties of one molecule of heteropolar compound with the hydrogen donor and acceptor moieties of another molecule of said heteropolar compound.

In general the preferred “nutrient” metal ion chelating agent is a hetero aryl compound having at least one nitrogen in the ring structure and at least one hydroxyl substituent disposed on the ring structure so as to provide together, a chelating function. Preferred metal ion chelating agents are selected from optionally substituted 2,3-dihydroxypyridine; 4,6-dihydroxypryrimidine; 2-pteridinol; 2,4-quinolindiol; 2,3-dihydroxyquinoxalin; 2,4-pteridinediol; 6-purinol; 3-phenanthridinol; 2-phenanthrolinol; 2-phenazinilol, and most preferred is 8-hydroxyquinoline. 8-hydroxyquinoline has the advantage of forming metal ion chelates with a particularly broad range of different metal ions.

In a further aspect the present invention provides a pharmaceutical composition for internal, external or injectable application comprising said primary and secondary chelating agents in a pharmaceutically acceptable carrier therefore, preferably an aqueous based carrier. Suitable aqueous based carriers will generally also include an intermediate diluent, wetting agent, a thickener where needed, and desirably, a pH controller. The compositions will generally be in the form of liquids, gels or pastes and will generally comprise from 0.0031% to 0.20% w/w, preferably from 0.02 to 0.1% w/w, of the (primary and secondary) chelating agents depending inter alia on the particular administration route used.

In the case of humans, for the control of tumours in the stomach, liver, and intestines, the composition is conveniently taken twice daily at the rate of 5 mls per dosage. This dosage is maintained until the cancerous tumour commences to shrink and then eventually disappear. For throat cancer, 10 mls of the aqueous mixture is taken in a glass of water and then gargled with.

For the control of isolated tumours then desirably the chelating agent composition is injected directly into the tumour. Depending on the particular location of the tumour, injection may advantageously be carried out by remote means with the aid of a suitable scanner to ensure that the mixture is injected into the proper area. An alternative is to install an infusion or syringe pump that will dispense into the tumour at predetermined intervals, the chelating solution over the required period of time. The period of injections or dosing required will generally be determined by suitable monitoring of the tumour to ensure that it is shrinking.

In the case of external tumours such as Karposi's tumours, open top tumours, blind tumours in humans or similar tumours in animals and sarcoids, which are prevalent in horses, there is advantageously used a paste formulation. The application rate is dependent on the location of the tumour on the body, and whether it is an open topped or blind tumour, with the latter requiring substantially higher dosage rates, and even then only achieving moderate success. With open top tumours such as sarcoids the paste formulation is generally applied twice a day for several days, typically 10 days. Within 6-8 weeks we have found that the sarcoids disappear leaving only a small bump on the skin where they had been.

In the case of oral cancer, for example, gum cancer, there is advantageously used a paste formulation which is rubbed onto the affected site, conveniently twice a day.

It will be appreciated that the choice of other components of the composition may be limited by the nature of the metal ion chelating agent. Thus, for example, since the preferred metal ion chelating agent 8-hydroxyquinoline is generally insoluble or only poorly soluble in aqueous solution. Suitable aqueous based compositions of 8-hydroxyquinoline can be prepared by using an intermediate solvent such as a polyolol, including glycols, preferably propylene glycol, glycerine, or sorbitol, and a wetting agent. Those skilled in the art will appreciate that a wide range of wetting agents are available that may be used which would give solubility of the metal ion chelating agent in glycol, including inter alia Polyoxyethylene Sorbitan Fatty Acid Ester T20, T40, T60 and T80 (Polysorbate), and C9-C11 Alcohol ethoxylate (Symperonic 91/8, or more preferably, Symperonic 91/6).

It will be appreciated that a range of different proportions of the various components of the aqueous based compositions may be used depending on the solubilities of the metal ion chelating agents used, the final concentration required etc. In general we have found that the amount of wetting agent used is relatively sensitive. In the case of the intermediate solvent (glycol etc), once a required minimum amount sufficient for solubilisation of the metal ion chelating agent in the water is present, then the amount of this intermediate solvent can be readily increased further, though there is normally no particular advantage in doing so.

Advantageously there is also included a pH controller, in order to ensure an alkaline pH in the composition, most preferably a pH in the region of 9.2 to 9.4. The pH controller may simply be KOH or NaOH. Preferably, though, there is used EDTA, conveniently in the form of the DiSodium or TetraSodium salt.

In the case of 8-hydroxyquinoline we have found that suitable proportions which may be used in a liquid, aqueous-based, composition of the invention suitable for use in the treatment of tumours, would in general have the following composition:

| Component | Weight |
|---|---|
| Primary chelating agent | 1 part |
| (8-hydroxyquinoline) | |
| Secondary chelating agent | 0.1 part |
| (EDTA) | |
| Wetting agent | 4 +/− 5% parts |
| Diluent | at least 20 preferably 40 |
| Deionised Water | as required to obtain |
| | the final concentration |
| | required. |
| pH controller (DSEDTA or TSEDTA) | as required to achieve a |
| | pH of 9.2 to 9.4. |

Further preferred features and advantages of the present invention will appear from the following detailed examples given by way of illustration of some preferred embodiments.

### EXAMPLE 1

### Method of Preparation of Concentrate

10 gm of 8-hydroxyquinoline (primary chelating compound) and 0.5 gram of Ethylene Diamine Tetra Acetic Acid (secondary chelating compound), were dissolved at 70 degrees centigrade in 50 grammes of a wetting agent selected from: Polyoxyethylene Sorbitan Fatty Acid Ester T20, T40, T60 and T80 (Polysorbate), and C9-C11 Alcohol ethoxylate (Symperonic 91/6), with 200 grams of a water soluble non-aqueous diluent selected from Propylene Glycol, Glycerine and Sorbitol. Once solution has been achieved, a further quantity of the glycol or glycerine or Sorbitol diluent was added to make up to a solution of 500 grammes and then cooled giving 500 g of a concentrate containing a mixture of 2.10% primary and secondary chelating compounds.

### EXAMPLE 2

### Preparation of Oral Composition

Take one part of the 2.1% chelating concentrate from Example 1 and dilute in 159 parts of deionised water, then the pH of this composition is adjusted to pH 9.2/9.4 by the addition of Tetra Sodium Ethylene Diamine Acetic Acid. The strength of this preparation is 131.25 ppm of chelating compounds making each dosage of 5 mls containing 656 microgram.

A suitable dosage rate of this composition is 10 mls per day giving a consumption of 1312 micrograms per day of chelating compounds, which—based on an average adult human body weight of 70 kg, would correspond to approximately 18.75 micrograms per kg body weight.

### EXAMPLE 3

### Preparation of Injectable Composition

Take one part of the 2.1% chelating concentrate from Example 1 and dilute in 319 parts of deionised water, then the pH of this composition is adjusted to pH 9.2/9.4 by the addition of Tetra Sodium Ethylene Diamine Tetra Acetic Acid. The strength of this preparation is 62.5 ppm of chelating agents making each injection quantity of 5 mls containing 312.5 micrograms.

Based on 2 injections per day each of 5 mls, this would provide a dosage rate of 625 micrograms a day of chelating compounds, which—based on an average adult human body weight of 70 kg, would correspond to approximately 8.93 micrograms per kg body weight.

This same solution can conveniently be administered by an infusion pump located externally or internally on a person's body, with dosage frequency and timing controlled by an external electronic unit. In this approach the dosage is automatic and the rate and frequency can be controlled externally.

### EXAMPLE 4

### Preparation of a Paste for External Tumours

Take one part of the 2.1% chelating concentrate from Example 1 and dilute in 9 parts of deionised water. A suitable thickener, normally Amaze XT, is then added at the rate of 2%, to produce a viscous paste and then the pH is adjusted in the composition to 9.2/9.4 with Tetra Sodium Ethylene Diamine Tetra Acetic Acid (TSEDTA).

The application of this paste is dependent on the tumour and the person concerned but a layer of the paste should be at least 2-3 mm thick on each application. It is necessary to keep the paste on the tumour moist so if necessary a suitable dressing or covering (conveniently in the form of disposable diaper material), is placed over the area with a plastic wrap around it, to prevent the paste from drying out.

### EXAMPLE 5

### Treatment of Horse Sarcoid Tumour

These typically are present in the folds between the legs and body, and in this case the paste tends to stay moist when applied twice daily. Treatment is typically for a period of 10 days.

1. A method of treatment or prophylaxis of a bacteria-mediated cancerous tumour or a tumour selected from Karposi and Sarcoid tumours, said method comprising the step of administering to a subject in need thereof, a therapeutically effective amount of a metal ion chelating agent wherein said metal ion chelating agent provides a metal ion chelating capacity for at least one metal ion on which said tumorigenic bacteria is dependent for viability.

2. (canceled)

3. The method according to claim 1 wherein said metal ion chelating agent is used together with a calcium ion chelating agent having a chelating capacity for calcium ions.

4. The method according to claim 1 wherein said metal ion chelating agent is a heteropolar compound comprising at least one unsaturated heterocyclic six-membered ring in which at least one heteroatom moiety acts as a hydrogen acceptor and wherein said heteropolar compound also comprises at least one hydrogen donor moiety, said heteropolar compound having no substituent which by itself or together with another substituent or substituents creates such steric hindrance and/or renders the molecule so basic or acidic or so alters the steric geometry of the molecule as to prevent interaction of the hydrogen donor and acceptor moieties of one molecule of heteropolar compound with the hydrogen donor and acceptor moieties of another molecule of said heteropolar compound.

5. The method according to claim 1 wherein said metal ion chelating agent is a hetero aryl compound having at least one nitrogen in the ring structure and at least one hydroxyl substituent disposed on the ring structure so as to provide together, a chelating function.

6. The method according to claim 5 wherein said metal ion chelating agent is selected from optionally substituted 2,3-dibydroxypyridine; 4,6-dibydroxypryrimidine; 2 pteridinol; 2,4-quinolindiol; 2,3- dibydroxyquinoxalin; 2,4 pteridinediol; 6-purinol; 3-phenanthridinol; 2 phenanthrolinol; 2-phenazinilol, and 8-hydroxyquinoline.

7. The method according to claim 6 wherein said metal ion chelating agent is 8-hydroxyquinoline.

8. The method according to claim 1 wherein said chelating agent is in a pharmaceutical formulation further comprising a wetting agent.

9. The method according to claim 8 wherein said wetting agent is selected from Polyoxyethylene Sorbitan Fatty Acid Ester T20, T40, T60 and T80 (Polysorbate), and C9-C11 Alcohol ethoxylate (including Symperonic 91/8, and Symperonic 91/6).

10. The method according to claim 1 wherein said chelating agent is in a pharmaceutical formulation further comprising an intermediate solvent in the form of a non-aqueous water soluble solvent.

11. The method according to claim 10 wherein said intermediate solvent is a polyol.

12. The method according to claim 11 wherein said intermediate solvent is selected from monoethylene glycol, propylene glycol glycerine, and sorbitol.

13. The method according to claim 1 further comprising a thickener.

14. The method according to claim 13 wherein said thickener is a hydroxypropylcellulose thickener.

15. The method according to claim 13 wherein said thickener is a dehydroxanthan gum thickener.

16. The method according to claim 1 comprising 1 part by weight of 8-hydroxyquinoline, about 3.95-4.05 parts by weight of wetting agent, at least 20 parts by weight of glycol, and water.

17. The method according to claim 1 wherein said chelating agent is in a pharmaceutical formulation in the form of a liquid, spray, cream, gel or paste.

18. The method according to claim 1 wherein said metal ion chelating agent is in a pharmaceutical formulation at a concentration of from 0.0031% to 0.20% w/w of the chelating agent(s).

19. The method according to claim 18 wherein said metal ion chelating agent is at a concentration of from 0.02 to 0.1% w/w of the chelating agent.

20. The method according to claim 1 wherein said chelating agent is in a pharmaceutical formulation further comprising a pH controller so that said pharmaceutical formulation has a pH in the range from 7.5 to 10.

21. The method according to claim 20 wherein said pharmaceutical formulation has a pH in the range from 9.2 to 9.4.

22. The method according to claim 3 wherein said calcium ion chelating agent comprises EDTA.

23. (canceled)

24. A method for the treatment or prophylaxis of a bacteria-mediated cancerous tumour, comprising the step of administrating to a subject in need thereof a therapeutically effective amount of at least one metal ion chelating agent, wherein said at least one metal ion chelating agent provides a metal ion chelating capacity for calcium ions and for at least one metal ion on which said tumorigenic bacteria is dependent for viability.
